(19) United States
(12) Patent Application Publication
(10) Pub. No.: US 2020/0261494 A1
(43) Pub. Date: Aug. 20, 2020

(54) SOLID ORAL FORMULATION AND METHOD TO TREAT CASES OF FLUID OVERLOAD IN ANIMALS

(71) Applicant: IsoTherapeutics Group LLC, Angleton, TX (US)

(72) Inventors: Jaime Simon, Angleton, TX (US); R Keith Frank, Lake Jackson, TX (US)

(73) Assignee: IsoTherapeutics Group LLC, Angleton, TX (US)

(21) Appl. No.: 16/759,955

(22) PCT Filed: Oct. 30, 2018
(86) PCT No.: PCT/US2018/058256

§ 371 Date: Apr. 28, 2020

Related U.S. Application Data
(60) Provisional application No. 62/579,996, filed on Nov. 1, 2017.

(51) Int. Cl. A61K 31/78; A23K 50/40; A61K 47/44; A23K 20/163; A23K 20/174; A23K 20/20; A23K 20/147; A61K 9/00

(52) U.S. Cl. CPC A61K 31/78; A23K 50/40; A61K 47/44; A61K 9/0056; A23K 20/174; A23K 20/30; A23K 20/147; A23K 20/163

(57) ABSTRACT

This invention relates to a solid veterinary formulation and a method for its oral administration to an animal, especially cats or dogs, to remove fluid overload in the animal by using a mixture of a water-absorbing polymer and solid fat, optionally having one or more supplemental ingredients. The fluid overload is caused by congestive heart failure or renal disease. The polymer is capable of absorbing at least 10 times its weight in physiological saline. The polymer and other waste materials are excreted in the feces.

### BACKGROUND OF THE INVENTION

### Field of the Invention

The invention relates a solid formulation having as its active ingredient a water-absorbing polymer and its method of use for treating animals having fluid overload.

### Description of Related Art

There are many serious disease states associated with the inability of the body to remove fluids. There are several reasons that can cause these conditions. One reason is when the kidneys fail to function correctly (total or partial renal failure), then removal of fluid by the kidneys is less than optimal. Another reason is if the heart does not pump the appropriate amount of blood in the system, then there is not enough blood pressure for the kidneys to remove water from the body. This can lead to a condition called congestive heart failure. These and other physiological problems can lead to fluid overload in the human or animal body. Several of these conditions are described in U.S. Pat. No. 8,263,112 which is incorporated herein by reference.

When such retention of fluid in the body occurs, there is a buildup of undesirable substances such as urea, creatinine, other nitrogenous wastes and electrolytes such as phosphate, potassium and calcium. Fluid overload is a serious condition that can cause severe symptoms and even death.

Restricting fluid intake and the use of diuretics are often used to treat fluid overload. However, restricting fluid intake does not relieve the build-up of toxins in the body. The use of diuretics to improve the body's ability to remove fluids can result in the loss of electrolytes such as sodium and potassium that are needed by the body. In addition, the effectiveness of diuretics can decrease with time. When diuretics do not work, dialysis may be used. Both hemodialysis and peritoneal dialysis are difficult procedures to administer and are very time consuming. In addition, both dialysis procedures are associated with significant side effects and decreased quality of life.

One approach has been the oral administration of crosslinked polyacrylate salts that was taught in Japanese Patent Application Kokai No. H10-59851, published Mar. 3, 1998 (Application No. H8-256387) and Japanese Patent Application Kokai No. H10-130154, published May 19, 1988 (Application No. H8-286446). The polymers were administered as a liquid oil emulsion to rats. This emulsion went directly into the stomach by gavage. These liquid emulsions are hard to handle and it is not easy to feed the emulsions directly to an animal. The active was in a capsule containing a polymer and oil that was taught to be orally administered to humans that required kidney dialysis. There is no data to show actual use in humans or animals other than rats.

U.S. Pat. No. 8,263,112 teaches that direct administration of a crosslinked polyacrylate polymer to the stomach of humans may degrade the polymer and that the polymer may absorb needed nutrients from the stomach and removes them from the body. For this reason the polymer was enteric-coated for the use taught. The polymers taught in that patent are incorporated herein by reference.

Due to the difficulty with handling liquid emulsions and the problems arising by delivering the polymer directly into the stomach, these liquid formulations are not desirable. In order to overcome these issues U.S. Pat. No. 8,263,112 teaches the use of an enteric-coating to prevent the exposure of the polymer to stomach contents. It teaches significant advantages to enteric-coating the polymer. However, enteric-coated super absorbent polymers are hard to make and are expensive.

Clearly, there is a need for a better treatment for fluid overload in animals.

### BRIEF SUMMARY OF THE INVENTION

The present invention provides a solid formulation of a polymer that can remove fluids and toxins from the body of animals with fluid overload diseases. The formulations of this invention include a polymer with the capability of absorbing large amounts of fluid per mass of polymer that, when incorporated into a solid or semi-solid matrix, can be administered orally. The preferred polymer is crosslinked polyacrylic acid in the form of its physiologically acceptable salts such as sodium or potassium. The polymer is formulated into a solid matrix that can be easily handled and administered orally. Preferred substrates used to form the solid final product include lard, butter, peanut butter, cheese or other fat-containing substrates that form a solid at room temperature. In addition, the polymer can be baked into cookies, bread, biscuits or other solid forms of baked goods. Alternatively, the polymer can be mixed into foods such as dog food that contain the correct fats described above and when combined with the polymers do not result in swelling. In addition, a combination of oils and fats can be used as long as the final formulation is a workable solid or semi-solid. Various flavorings can be added to the formulation. The solid formulations containing the polymer are easy to handle and can be easily packaged.

### DETAILED DESCRIPTION OF THE INVENTION

It is understood that the terminology used herein is for the purpose of describing particular embodiments only and is not intended to be limiting. As used in this specification, the singular forms “a”, “an”, and “the” include plural referents unless the content clearly indicates otherwise. The following terms in the Glossary as used in this application are to be defined as stated below and for these terms, the singular includes the plural.

Various headings are present to aid the reader, but are not the exclusive location of all aspects of that referenced subject matter and are not to be construed as limiting the location of such discussion.

Also, certain US patents and PCT published applications have been incorporated by reference. However, the text of such patents is only incorporated by reference to the extent that no conflict exists between such text and other statements set forth herein. In the event of such conflict, then any such conflicting text in such incorporated by reference US patent or PCT application is specifically not so incorporated in this patent.

### Glossary

% means weight percent, unless stated otherwise.
Animal means any warm-blooded animal or mammal, excluding humans.
Dosage means the mass of polymer administered which is enough mass of polymer to cause reduction of the fluid overload in the animal which is on average about 0.25 to about 4 g per pound body weight; more preferred 1 to 2 g per pound body weight. The dosage may also be altered with the severity of the condition. For example, a severely fluid overloaded animal may require 3-4 grams of polymer per pound of body weight. However, a less severe condition or a maintenance dose may be 0.5 or less grams of polymer per pound of body weight.
Fat means a solid at room temperature (RT) which are mostly saturated fats found, for example in beef fat, butter or shortening, and can be made from vegetable oils by hydrogenation. These solids fats are triglycerides or triacylglycerols that are saturated and a solid.
GRAS means Generally Recognized As Safe under §§ 201(s) and 409 of the Federal Food, Drug, and Cosmetic Act (US FDA), that is any substance that is intentionally added to food is considered a food additive, that is subject to premarket review and approval by US FDA, unless the substance is generally recognized, among qualified experts, as having been adequately shown to be safe under the conditions of its intended use, or unless the use of the substance is otherwise excluded from the definition of a food additive.

RT means room temperature that is ambient temperature, about 20-25° C.

Water Absorbent Polymer means a non-systemic, non-toxic, non-digestible, fluid absorbing polymer such as crosslinked polyacrylates and those described below.

The present invention consists of a formulation that is a solid at room temperature and is a mixture of a water absorbent polymer and a solid fat wherein the ratio of polymer to fat is from about 40% by weight of polymer to 60% by weight of fat to about 60% by weight of polymer to 40% by weight of fat, preferably to about 50:50% by weight. The supplemental ingredients when present are usually from about 0.5% by weight to 5% by weight of the total formulation. These solid formulations contain a water absorbent polymer capable of absorbing fluid from the body into the GI tract and removing the undesirable fluid with the fecal matter. These solid formulations are easy to handle and store. They are particularly useful for treating animals such as dogs and cats that are afflicted with fluid overload from either renal disease or congestive heart failure.

A treatment for veterinary purposes for fluid overload is needed that can be administered easily. Dialysis is not practical or desirable and cost prohibitive for animals. Rather the present invention is a treatment using a water-absorbent polymer that can absorb fluids in the GI tract and excrete them in the feces thereby relieving fluid overload. Because the water-absorbent polymer binds fluid and is excreted in the feces, this process relieves kidney excretion load. Surprisingly, it has been found that no enteric coating of the water-absorbent polymer is required.

The water-absorbent polymer includes: crosslinked polymers such as those prepared from α,β-ethylenically unsaturated monomers such as monocarboxylic acids, polycarboxylic acids, acrylamide and their derivatives, e.g., polymers having repeating units of acrylic acid, methacrylic acid, metal salts of acrylic acid, acrylamide, and acylamide derivatives (e.g., 2-acrylamido-2-methylpropane-sulfonic acid) along with various combinations of such repeating units as copolymers. Such derivatives include acrylic polymers which include hydrophilic grafts of polymers such as polyvinyl alcohol. Examples of suitable polymers and processes, including gel polymerization processes for preparing such polymers are disclosed in U.S. Pat. Nos. 3,997,484; 3,926,891; 3,935,099; 4,090,013; 4,093,776; 44,340,706; 4,446,261; 4,683,274; 4,459,396; 4,708,997; 4,076,663; 4,190,562; 4,286,082; 4,857,610; 4,985,518; 5,145,906; and 5,629,377, which are incorporated herein by reference. Furthermore a text by Buchholz, F. I, et al., “Modem Superabsorbent Polymer Technology”, pub. John Wily &Sons (1998) also teaches polymers useful in this invention. For this invention the water-absorbent polymer is lightly crosslinked (e.g., less than 0.2 mole percent crosslinking agent is included) such that the polymer can still absorb over 10 times its weight in physiological saline (i.e. 0.9% saline) and some polymers can absorb 20, 30 or 40 times their weight. The morphological form is not significant. Food or pharmaceutical grades of these water absorbent polymers are preferred for this invention. When additional waste products need to be removed from the body, these polymers may be functionalized with groups that will selectively bind with these waste products, such functionalized groups include aldehyde groups for binding urea, polyaminoalkylene groups for binding oxalate, such as triethylenetetramine or tetraethylenepentamine, or amino functional groups for binding phosphate or oxalates.

Various additional ingredients can be included in the formulation, as desired, especially GRAS ingredients and FDA approved food additives. For example, such ingredients are: sweeteners, either sugar or low calorie sweeteners; a food approved antioxidant; water-soluble vitamins; emulsified lipid-soluble vitamins; mineral supplements; gelatin, agar-agar, carrageenan, pectin; cocoa powder, caffeine; amino acids; maltodextrin; and flavorings such as licorice, fruit, spice or other flavorings. The formulation should be in a form acceptable to the animal and have a flavor to assist in the polymer dose being consumed.

One such ingredient is one or more low calorie sweeteners such as xylitol, sorbitol, or an artificial sweetener or mixture thereof. Some examples of such sweeteners are: aspartame (e.g., Equal®, trademark of Merisant Company) and/or sucralose (e.g., Splenda®, trademark of McNeil Nutritionals, LLC, which is a sucralose-based artificial sweetener derived from sugar, blended with maltodextrin) and/or stevia (e.g., Truvia®, a trademark of The Coca-Cola Company, which is a low caloric sweetener derived from the stevia plant and blended with erythritol), or 99% pure Rabaudioside A, also derived from Stevia Rebaudiana leaves (e.g., Good & Sweet® trademark of Blue California). Xylitol is a low caloric naturally occurring sweetener, used in chewing gums, and is classified as a GRAS substance. Sucralose and aspartame are well-known artificial sweeteners. Additionally, when desired sugar can be the sweetener used if it poses no issue for the fluid overload of the animal being treated.

Another optional ingredient that can be added to the present formulation is an antioxidant to provide taste improvement or preservation properties as to its taste. Any approved food antioxidant can be used such as ascorbic acid (vitamin C), citric acid, sodium benzoate and others.

Various flavorings can be added to the present formulation for the taste desired, such as licorice, peanut butter, bacon, cocoa, coffee, tea flavors; fruit flavorings such as lime, lemon, orange, cherry, strawberry, peach, mixed berry, pomegranate; and spices, such as cinnamon, nutmeg, ginger, chamomile, ginseng, anise, pumpkin and others.

These formulations can be administered to an animal to treat fluid overload. The amount administered (dose) and the frequency (number of time per day) will depend on many factors such as the usual amount of renal excretion, the amount of water consumed, the polymer being used, and if other waste products or toxins need to be removed. Some preferred doses and frequencies are: about 0.25 to about 4 g per lb. of the animal; e.g., about 30 g to about 60 g daily for a 70 lb. dog. The amount of water-absorbent polymer present in the total formulation is about 40 to 60%.

### DISCUSSION

The invention will be further clarified by a consideration of the following examples, which are intended to be purely exemplary of the invention.

Materials and Equipment:

The Water Absorbent Polymer was purchased from Sigma Aldrich and is a crosslinked polyacrylate (Cat No. 432784) or purchased as soil moist granules from Rootnaturally.com.

The pharmaceutically acceptable components of the formulation are FDA approved food grade additives or GRAS and purchased commercially.

General Procedure

In the following examples, the lettered examples are comparative, and the numbered examples are this invention.

### Example A: Comparative—Cream Cheese (Philadelphia Cream Cheese® Trademark of KraftHeinz Food Company)

One gram of polymer was added into 1 gram of cream cheese. The solution did not mix well and was not improved with heat. The reaction swelled implying that the water content of the sample was too high.

### Example B: Comparative—Cottage Cheese (Kroger® Trademark of the Kroger Co. of Michigan)

One gram of polymer was added to 1 gram of cottage cheese. The components did not mix at all and large chunks of cheese remained visible. Heat did not improve texture, and the polymer started to swell.

### Example C: Comparative—Greek Yogurt (Dannon® Trademark of DANONE US, LLC LIMITED LIABILITY COMPANY)

One gram of polymer was added to 1 gram of Greek yogurt. The two components did not mix well, but consistency improved with heat. The polymer swelled.

### Example 1: Lard (Crisco® Trademark of THE J. M. SMUCKER COMPANY CORPORATION)

One gram of polymer was mixed with I gram of lard in a scintillation vial. The mixture was heated until it was mixed well. After cooling to room temperature, the formulation turned into a solid that was easy to handle. The polymer did not swell.

### Example 2: Butter (Kroger@ trademark of The Kroger Co. of Michigan)

One gram of polymer was mixed with I gram of butter. The mixture was heated until it mixed well. After cooling to room temperature, the mixture turned into a solid that was easy to handle. The polymer did not swell.

### Example 3: Peanut Butter (Peter Pan® Creamy, Trademark of CONAGRA FOODS RDM, INC.)

One gram of polymer was mixed with I gram of peanut butter. The polymer mixed well into the peanut butter without heating. The final product was a solid that was easy to handle. The polymer did not swell.

### Example 4: Cookies

In a large 1500 mL beaker, 125 grams of flour, 5 grams of baking powder, and 1 egg were mixed together. A volume 236 mL of water, and 29.4 mL of honey were later added to the mixture. The total volume was around 400 mL. Two 200 mL beakers were filled with 50 mL each of the solution. Beaker A was designated to contain the polymer/fat mixture, while beaker B contained no polymer (comparative). A mass of 37 grams of peanut butter and 10 grams of polymer were mixed together and added to beaker A. A mass of 38 grams of peanut butter alone were added to beaker B. The contents were mixed thoroughly, rolled, and placed on a baking sheet to be baked at 350° F. for 15 minutes.

Both formulations formed solid materials that resembled cookies. Surprisingly, the dough containing polymer did not swell before or after baking. After baking, a portion of a polymer-containing cookie was crumbled into a plate and water added. The polymer absorbed the water as well as if it was not formulated into the cookie.

The final polymer-containing cookies were fed to a dog with congestive heart failure. The cookie formulation was well liked by the dog and ingested rapidly. The fecal matter from the dog showed swelled polymer indicating that it absorbed fluids and removed them from the body.

### Example 5: Congestive Heart Failure

A 14.5-year-old female rat terrier (patient dog) was diagnosed with congestive heart failure by a veterinarian two years prior. The patient dog was visibly fluid overloaded due to ascites, not as active, having trouble climbing stairs and getting up repeatedly through the night. Her condition further deteriorated and she became lethargic to the point of being non-ambulatory. She even began refusing food.

Lightly crosslinked polyacrylic acid (super absorbent polymer) was combined with canned cat food, cooked chicken or scrambled eggs (to entice her to eat) and fish oil to coat the polymer and mix well with the food in order to make the polymer in a form that the dog could swallow and not choke. The patient dog was given orally a dose of about 15 g of polymer twice daily. She tolerated the dose well and her feces was found to contain fluid swelled polymer showing that the polymer was removing fluids from the body. Within a week her abdominal swelling was significantly reduced, and the patient dog became significantly more active. Her appetite returned, she became able to navigate stairs and returned to an activity level that she had not had for several years. After several months the dose of the polymer was reduced to about 15 grams per day without any signs of regression. The patient dog was finally reduced to a maintenance dose of about 7 g of polymer a day and has survived for two years without any signs of toxicity from the treatment. After two years, the patient dog is still alive living a normal and very active life at the age of 17.

Although the invention has been described with reference to its preferred embodiments, those of ordinary skill in the art may, upon reading and understanding this disclosure, appreciate changes and modifications which may be made which do not depart from the scope and spirit of the invention as described above or claimed hereafter. Accordingly, this description is to be construed as illustrative only and is for the purpose of teaching those skilled in the art the general manner of carrying out the invention.

1. A veterinary formulation comprising a non-enterically coated, water-absorbing polymer in a solid mixture, and optionally containing one or more supplemental ingredients, wherein the resulting formulation has about 40% to about 60% by weight of the water-absorbing polymer in the total formulation and is an oral solid or semi-solid formulation.

2. The formulation of claim 1, wherein the water-absorbing polymer is mixed with a solid fat.

3. The formulation of claim 1, wherein the polymer is mixed with an oil or fat and another excipient resulting in a solid or semi-solid.

4. (canceled)

5. The formulation of claim 1, wherein the supplemental ingredients are food additives, including flavorings, and GRAS ingredients from about 0.5% by weight to 5% by weight of the total formulation.

6. The formulation of claim 5, wherein such supplemental ingredients are one or more sweeteners; a food approved antioxidant; water-soluble vitamins; emulsified lipid-soluble vitamins; mineral supplements; gelatin, agar-agar, carrageenan, pectin; cocoa powder; caffeine; amino acids; maltodextrin: or flavorings.

7. The formulation of claim 5, wherein the supplemental ingredients are flavorings selected from licorice, peanut butter, bacon, cocoa, coffee, tea flavors, lime, lemon, orange, cherry, strawberry, peach, mixed berry, pomegranate, cinnamon, nutmeg, ginger, chamomile, ginseng, anise, or pumpkin.

8. The formulation of claim 1, wherein the water-absorbing polymer is capable of absorbing at least 10 times its weight in physiological saline.

9. The formulation of claim 8, wherein the water-absorbing polymer is capable of absorbing at least 20, 30 or 40 times its weight in physiological saline.

10. The formulation of claim 1, wherein the water-absorbing polymer is formed by polymerizing acrylate-containing monomers.

11. The formulation of claim 1, wherein the water-absorbing polymer is formed by polymerizing a monomer comprising acrylic acid or salts thereof.

12. The formulation of claim 1, wherein the water-absorbing polymer is crosslinked polyacrylate.

13. A method for treating an animal having fluid overload from congestive heart failure or renal disease comprising administering an effective amount of a solid or semi-solid formulation of claim 1, wherein the water absorbent polymer is capable of absorbing at least 10 times its weight in physiological saline.

14. The method of claim 13, wherein the animal is a cat or dog.

15. The method of claim 13, wherein the effective amount administered is about 0.25 to about 4 g per pound of the animal.

16. The method of claim 13, wherein the effective amount administered is about 2 g to about 60 g daily for a 70-pound dog.

17. The method of claim 13, wherein the solid or semi-solid formulation is a formulation of claim 2.

18. The method of claim 13, wherein the solid or semi-solid formulation is a formulation of claim 3.

19. The method of claim 13, wherein the solid or semi-solid formulation was administered to the animal 1 or 2 times per day.

20. The formulation of claim 1, wherein the water-absorbing polymer has been further functionalized by aldehyde groups, poly(aminoalkylene) groups, or amino functional groups.
